(19) United States
(12) Patent Application Publication
(10) Pub. No.: US 2009/0110744 A1
(43) Pub. Date: Apr. 30, 2009

(54) SUSTAINED RELEASE PHARMACEUTICAL COMPOSITIONS

(75) Inventors: Ajay Khopade, Baroda (IN); Alex George, Baroda (IN); Subhas Balaram Bhowmick, Baroda (IN)

Correspondence Address: SUGHRUE MION, PLLC, 2100 PENNSYLVANIA AVENUE, N.W., SUITE 800, WASHINGTON, DC, 20037, US

(73) Assignee: SUN PHARMA ADVANCED RESEARCH COMPANY LIMITED, Mumbai (IN)

(21) Appl. No.: 11/817,592

(22) PCT Filed: Mar. 1, 2006
(86) PCT No.: PCT/IN06/00066

§ 371 Date: May 13, 2008

(30) Foreign Application Priority Data
Mar. 1, 2005 (IN) 232/MUM/2005

(51) Int. Cl. A61K 9/14; A61K 38/08

(52) U.S. Cl. 424/501; 514/16

(57) ABSTRACT

A sustained release microsphere composition comprising—
(i) microspheres comprising (A) a biodegradable polymer which is a homopolymer of lactic acid or a copolymer of lactic acid and glycolic acid having a monomer ratio in the range of about 1:1 to about 3:1, and
(B) a therapeutically effective amount of octreotide acetate, and
(ii) pharmaceutically acceptable excipients,
   which when injected, delivers octreotide acetate, for a period of at least one month.

### FIELD OF THE INVENTION

The present invention relates to sustained release pharmaceutical microsphere compositions comprising octreotide acetate.

### BACKGROUND OF THE INVENTION

Octreotide is the acetate salt of a cyclic octapeptide. It is a long-acting octapeptide with pharmacological properties mimicking those of the natural hormone somatostatin. Octreotide is known chemically as L-Cysteinamide, D-phenylalanyl-L-cysteinyl-L-phenylalanyl-D-tryptophyl-L-lysyl-L-threonyl-N-[2-hydroxy-1-(hydroxymethyl)propyl]-, cyclic (2->7)disulfide; [R-(R*,R*)]. Octreotide acetate is indicated to reduce blood levels of growth hormone and IGF-I (somatomedin C) in acromegaly patients who have had inadequate response to or cannot be treated with surgical resection, pituitary irradiation, and bromocriptine mesylate at maximally tolerated doses. It is indicated for the symptomatic treatment of patients with metastatic carcinoid tumors where it suppresses or inhibits the severe diarrhea and flushing episodes associated with the disease. It is further indicated for the treatment of the profuse watery diarrhea associated with VIP-secreting tumors. Octreotide is available in the US as Sandostatin® (octreotide acetate) Injection and Sandostatin LAR® Depot (octreotide acetate for injectable suspension). Both the Sandostatin® Injection and Sandostatin LAR® Depot are marketed in the US by Novartis. Sandostatin® Injection is prepared as a clear sterile solution of octreotide, acetate salt, in a buffered lactic acid solution for administration by deep subcutaneous (intrafat) or intravenous injection. Sandostatin LAR® Depot is available in a vial containing the sterile drug product, which when mixed with diluent, becomes a suspension that is given as a monthly intragluteal injection.

### OBJECT OF THE INVENTION

It is an object of the invention to provide sustained release pharmaceutical microsphere compositions comprising octreotide acetate.

It is another object of the present invention to provide a sustained release microsphere composition comprising octreotide acetate, suitable for intramuscular administration and capable of sustaining release for a prolonged period of one month or three months or six months or more.

### SUMMARY OF THE INVENTION

The present invention relates to a sustained release microsphere composition of octreotide acetate and provides in its various embodiments the following:
(a) A sustained release microsphere composition comprising—
   (i) microspheres comprising (A) a biodegradable polymer which is a copolymer of lactic acid and glycolic acid having a monomer ratio in the range of about 1:1 to about 3:1, and (B) a therapeutically effective amount of octreotide acetate, and
   (ii) pharmaceutically acceptable excipients.
(b) A sustained release microsphere composition as described in (a) above, wherein the biodegradable polymer used has an average molecular weight within the range of about 10,000 to about 20,000.
(c) A sustained release microsphere composition as described in (a) above, wherein the microspheres have a volume mean diameter in the range of about 10 microns to about 20 microns.
(d) A sustained release microsphere composition as described in (a) above, wherein the composition is capable of delivering octreotide acetate over a period of about one month or about three months or about six months.
(e) A sustained release microsphere composition as described in (a) above, wherein the microsphere is suitable for intramuscular injection.
(f) A sustained release microsphere composition as described in (e) wherein the composition is capable of delivering octreotide acetate for a period of about one month or about three months or about six months.
(g) A sustained release microsphere composition as described in (a) above, wherein the pharmaceutically acceptable excipient is mannitol.
(h) A sustained release microsphere composition comprising—
   (i) microspheres comprising (A) a biodegradable polymer which is a homopolymer of lactic acid or a copolymer of lactic acid and glycolic acid having a monomer ratio in the range of about 1:1 to about 3:1, and (B) a therapeutically effective amount of octreotide acetate, and
   (ii) pharmaceutically acceptable excipients,
which when injected, delivers octreotide acetate, for a period of at least one month.

### DETAILED DESCRIPTION OF THE PRESENT INVENTION

The present invention relates to a sustained release microsphere composition comprising—
(i) microspheres comprising (A) a biodegradable polymer which is a homopolymer of lactic acid or a copolymer of lactic acid and glycolic acid having a monomer ratio in the range of about 1:1 to about 3:1, and (B) a therapeutically effective amount of octreotide acetate, and
(ii) pharmaceutically acceptable excipients,
which when injected, delivers octreotide acetate, for a period of at least one month.

The prolonged release microsphere of the present invention is made by preparing a water-in-oil emulsion comprising a first dispersed phase containing octreotide or a pharmaceutically acceptable salt thereof and an active ingredient-retaining substance therefor, and an outer phase containing a biodegradable polymer, followed by thickening or solidifying said first dispersed phase to a viscosity of not lower than about 5000 centipoises, and finally subjecting the resulting emulsion to a drying process.

The microspheres of the present invention may be prepared by the process described in co-pending applications 231/MUM/2005 and 1182/MUM/2005, the contents of which are incorporated herein by reference.

These applications provide a process for the preparation of free-flowing uniformly sized microspheres or microcapsules for the sustained release of therapeutically active ingredient, the process comprising:
a. preparing a first dispersed phase comprising a therapeutically active ingredient, a biodegradable polymer and an organic solvent;
b. mixing the first dispersed phase with an aqueous phase to form an emulsion;
c. spraying the emulsion into a vessel equipped with organic solvent removal means.
d. passing the suspension of microspheres or microcapsules through a first screen to remove large sized microspheres or microcapsules having a size greater than the mesh size of the first screen and then through a second screen to remove microspheres or microcapsules having a size smaller than the mesh size of the second screen, thereby collecting a fractionated size of the microspheres or microcapsules on the surface of the second screen;
e. drying the microspheres or microcapsules,
wherein steps a to e are carried out without manual intervention, in equipment connected in series, substantially unexposed to the environment.

In the above process, the drying step comprises lyophilization, freeze-drying, or air-drying the microspheres or microcapsules.

These applications also provide a process for the preparation of a lyophilized composition for the sustained release of a therapeutically active ingredient, the process comprising:
a. preparing a first dispersed phase comprising a therapeutically active ingredient, a biodegradable polymer and an organic solvent;
b. mixing the first dispersed phase with an aqueous phase to form an emulsion;
c. spraying the emulsion into a vessel equipped with organic solvent removal means to prepare a suspension of microspheres or microcapsules in a liquid vehicle;
d. passing the suspension of microspheres or microcapsules through a first screen to remove large sized microspheres or microcapsules having a size greater than the mesh size of the first screen and then through a second screen to remove microspheres or microcapsules having a size smaller than the mesh size of the second screen, thereby collecting a fractionated size of the microspheres or microcapsules on the surface of the second screen;
e. drying the microspheres or microcapsules;
f. suspending the microspheres or microcapsules in aqueous solution of a stabilizer,
g. transferring the suspension comprising the microspheres or microcapsules and the stabilizer into shallow freeze-drying container;
h. subjecting the suspension to lyophilization and dry-powder filling the lyophilized composition into unit dose containers,
wherein steps a to e are carried out without manual intervention, in equipment connected in series, substantially unexposed to the environment.

In the microspheres or microcapsules of the present invention, the active ingredient is present in a first dispersed phase along with the biodegradable polymer and an organic solvent. Depending on the active ingredient and depending on the polymer that may be used, the first dispersed phase may be a solution or an emulsion. If the active ingredient is water-soluble, then it is typically dissolved in a minimal quantity of purified water, while the biodegradable polymer is dissolved in a suitable organic solvent. These two solutions are then emulsified to obtain the first dispersed phase. Alternatively, if the active ingredient is water-insoluble, then it is dissolved in the organic solvent along with the biodegradable polymer to obtain the first dispersed phase. In the process of the present invention, when the first dispersed phase used is a solution, then microspheres are produced by the process of the invention, whereas when the first dispersed phase used is an emulsion, microcapsules are produced by the process of the invention. For the purposes of this application, the terms microcapsule and microsphere can be used interchangeably and the term “microsphere” is used throughout this application for the sake of convenience.

The sustained release microsphere composition of the present invention uses octreotide or a pharmaceutically acceptable salt thereof, preferably the acetate salt, as the active therapeutic ingredient. Preferably the octreotide acetate may be present in amounts ranging from the equivalent of about 0.1 mg to about 30 mg of octreotide base per vial.

The active ingredient-retaining substance employed in accordance with the present invention is either a substance which is soluble in water and hardly soluble in the organic solvent contained in said oil layer and when dissolved in water assumes a viscous semi-solid consistency or a substance which gains considerably in viscosity to provide a semi-solid or solid matrix under the influence of an external factor such as temperature pH, metal ions (e.g., Cu++, Al+++, Zn++, etc.), organic acids (e.g., tartaric acid, citric acid, tannic acid, etc.), a salt thereof (e.g., calcium citrate, etc.), chemical condensing agents (e.g., glutaraldehyde, acetaldehyde), etc. As examples of such active ingredient retaining substance may be mentioned natural or synthetic mucilages and high molecular weight compounds. Among such natural mucilages are gum acacia, Irish moss, gum karaya, gum tragacanth, gum guaiac, gum xanthan, locust bean gum, etc., while natural high molecular weight compounds include, among others, various proteins such as casein, gelatin, collagen, albumin (e.g., human serum albumin), globulin, fibrin, etc. and various carbohydrates such as cellulose, dextrin, pectin, starch, agar, mannan, etc. These substances may be used as they are or in chemically modified forms, e.g., esterified or etherified forms (e.g., methylcellulose, ethylcellulose, carboxymethylcellulose, gelatin succinate, etc.), hydrolyzed forms (e.g., sodium alginate, sodium pectinate, etc.) or salts thereof As examples of said synthetic high molecular weight compounds may be mentioned polyvinyl compounds (e.g., polyvinyl pyrrolidone, polyvinyl alcohol, polyvinyl methyl ether, polyvinyl ether, etc.), polycarboxylic acids (e.g., polyacrylic acid, polymethacrylic acid, Carbopol [Goodrich & Co., U.S.A.], etc.), polyethylene compounds (e.g., polyethylene glycol, etc.) and polysaccharides (e.g., polysucrose, polyglucose, polylactose, etc.) and salts thereof Also included are those compounds which undergo condensation or cross-linking under the influence of said external factors to give molecular weight compounds. Among the aforementioned compounds, gelatin, albumin, pectin and agar are particularly desirable. These compounds may be used alone or in combination and while the proportion of such compounds depends on the kind of compound, it is selected from the range of about 0.05% to 80% (w/w) in terms of concentration in the first dispersed phase, preferably from the range of about 0.1% to 50% (w/w) on the same basis. It should, however, be understood that such compounds must be used in sufficient amounts to ensure that the initial viscosity of the first dispersed phase in the water-in-oil emulsion described hereinafter will be not lower than about 5000 centipoises (cps), preferably not lower than about 10000 cps, or the first dispersed phase may be increased in viscosity to not lower than about 5000 cps, preferably not lower than about 10000 cps, or be solidified by external factors.

The present invention uses suitable biodegradable polymers such as polylactide polymers. The term “polylactide” is used in a generic sense to include polymers of lactic acid alone, copolymers of lactic acid and glycolic acid, mixtures of such polymers, mixtures of such copolymers, and mixtures of such polymers and copolymers, the lactic acid being either in racemic or in optically active form. The polylactide copolymers used in the present invention may have a ratio of lactic acid and glycolic acid in the range of about 1:1 to about 1:0. Preferably the present invention uses a homopolymer of lactic acid or a copolymer of lactic acid and glycolic acid (PLGA) having a monomer ratio in the range of about 1:1 to about 3:1.

The average molecular weight of such a biodegradable polymer as used in accordance with this invention ranges from about 2,000 to about 8,00,000 Daltons and is desirably selected from the range of about 5,000 to about 2,00,000 Daltons. Preferably the average molecular weight of the polylactide biodegradable polymer used in the present invention may range from about 5,000 to about 100,000 Daltons. In a preferred embodiment, the average molecular weight of the polylactide biodegradable polymer used is in the range from about 5,000 to about 30,000 Daltons. In a most preferred embodiment, the average molecular weight of the polylactide biodegradable polymer used in the present invention is in the range of about 10,000 to about 20,000 Daltons.

The polylactide polymer used in the present invention is used in amounts ranging from about 70% to about 99% W/W of the microspheres. This range of the amount of the polymer is used when about 1% to about 30% W/W of the active ingredient is loaded into the microspheres. Also, this amount of the polymer is calculated for the microspheres comprising the active ingredient, the polylactide polymer and the active ingredient retaining substance, but not the other pharmaceutical excipients used for suspending the microspheres before lyophilization. In an embodiment of the present invention, the polylactide polymer is used in amounts ranging from about 88% to about 90% W/W of the microspheres, when about 10% to about 12% W/W of the active ingredient is loaded in the microspheres. The proportion of such a biodegradable polymer depends on the strength of pharmacological activity of the therapeutically active ingredient used and the rate and duration of release of the active ingredient. By way of illustration, the proportion of this biodegradable polymer may range from ⅕ to 10000 times and preferably 1 to 1000 times the weight of the water-soluble active ingredient.

The solution containing said biodegradable polymer (oil layer) is a solution of the polymer in a solvent. The solvent for this purpose should be one which boils at a temperature up to about 120° C., is immiscible with water and capable of dissolving the polymer, and as such there may be mentioned halogenated alkanes (e.g., di-chloromethane, chloroform, chloroethane, dichloroethane, trichloroethane, carbon tetrachloride, etc.), ethyl acetate, ethyl ether, cyclohexane, benzene, n-hexane and toluene. These solvents may be used alone or in combination. Typically, the solvents are used in minimum amounts.

With regard to the microencapsulation procedure, the active ingredient-retaining substance in an amount sufficient to give the aforementioned concentration is first dissolved in water and, then, the water-soluble active ingredient is added in an amount sufficient to give the aforementioned concentration, whereby a first dispersed layer is provided. As a pH-adjusting agent for maintaining the stability and solubility of the water-soluble active ingredient, there may be incorporated in this first dispersed layer such an additive as carbonic acid, acetic acid, oxalic acid, citric acid, tartaric acid, succinic acid or phosphoric acid, sodium or potassium salts thereof, hydrochloric acid or sodium hydroxide. Moreover, as a stabilizer for the water-soluble active ingredient, there may also be added such an agent as albumin, gelatin, citric acid, ethylenediamine sodium tetraacetate, dextrin, sodium hydrosulfite, etc. The first dispersed phase may also contain a preservative such as p-oxybenzoic acid esters (e.g., methylparaben, propylparaben, etc.), benzyl alcohol, chlorobutanol, thimerosal, and the like. The first dispersed phase is emulsified using a solution of the polymer in a first tank, to obtain a primary water-in-oil emulsion. The emulsification can be effected by the conventional dispersion techniques. For example, intermittent shaking, mixing by means of a propeller mixer, turbine mixer or the like, colloid mill operation, mechanical homogenization, ultrasonication, and the like may be utilized.

When the viscosity of the first dispersed layer in such a water-in-oil emulsion is more than about 5000 centipoises or preferably over about 10000 centipoises from the beginning, the emulsion is immediately subjected to a evaporation procedure but, otherwise, resort is had to an external factor to thicken the first dispersed phase to a viscosity over about 5000 centipoises or preferably over about 10000 centipoises or solidify the same. Exemplary procedures for increasing the viscosity include a heat treatment, cooling to a low temperature, freezing, rendering the pH acidic or alkaline, or adding such an agent as metal ions (e.g., iron ion for gum acacia, copper ion for carboxymethylcellulose, or calcium or magnesium ion for sodium pectinate) or organic acids or salts thereof (e.g., calcium citrate for sodium alginate, or adipic acid or tartaric acid for polyvinyl alcohol). There may also be mentioned the technique of cross-linking and condensing the biodegradable polymer in the first dispersed phase using a chemical condensing agent (e.g., glutaraldehyde, acetaldehyde, etc.) With regard to the heat treatment, the procedure must be carried out in a closed vessel so as to avoid evaporation of the solvent contained in the oil layer. The temperature is virtually optional only if it is higher than the gelation temperature. This treatment thickens or solidifies the first dispersed phase. The technique of cooling the emulsion to a low temperature comprises cooling it to about −5° C. to about 35° C. and maintaining the low temperature with stirring for about 1 minute to about 6 hours. In the case of agar whose gelation point is about 40° C., the emulsification is conducted under heating at about 50° to 80° C. and, then, caused to gel at the above-mentioned temperature. For all types of first dispersed phase, it may be frozen by cooling at about −60° C. to 0° C. but the temperature should not be below the solidification point of the oil layer. As regards the procedure of adding a metal ion, an organic acid or a salt thereof, the amount thereof depends on the amount of the active ingredient retaining substance in the first dispersed phase and may range from about ¼ to 20 molar equivalents and preferably from about 1 to 10 molar equivalents. The time required for said thickening or solidification is preferably not more than about 6 hours. With regard to the technique of cross-linking and condensing the high molecular compound in the first dispersed phase with chemical condensing agent, such condensing agent may for example be an aqueous solution of glutaraldehyde or acetaldehyde or a solution of the same in an organic solvent such as halogenated alkanes (e.g., chloroform, dichloromethane, etc.), toluene, etc. Particularly, a solution in the latter solvent which is miscible with the solvent used in the oil layer is desirable, because the particle size of the first dispersed phase is not increased. The chemical condensing agent is added in a proportion of about 2 to 5 molar equivalents based on the active ingredient retaining substance in the first dispersed phase and the mixture is reacted under stirring for about 1 to 10 hours. More specifically, taking gelatin as an example of said active ingredient retaining substance, a water-in-oil emulsion of predetermined particle size is first prepared and then cooled to about 0° to 10° C. for about 5 to 30 minutes with constant stirring, whereby the first dispersed phase is caused to gel into semi-solid consistency. The water-in-oil emulsion thus prepared is subjected to in water drying. Thus, this water-in-oil emulsion is added to a third aqueous layer to give a W/O/W ternary emulsion and, finally, the solvent in the oil layer is desorbed to give microcapsules.

The second phase is typically an aqueous solution of an emulsifying agent that assists in the formation of the final O/W or W/O/W emulsion. The second phase is prepared by simply dissolving the emulsifying agent in purified water under aseptic conditions. Examples of the emulsifying agents that may be used include, but are not limited to, anionic surfactants (e.g., sodium oleate, sodium stearate, sodium laurylsulfate, and the like), nonionic surfactants (e.g., polyoxyethylene sorbitan fatty acid esters [Tween 80 and Tween 60, Atlas Powder, U.S.A.], polyoxyethylene castor oil derivatives [HCO-60 and HCO-50, Nikko Chemicals, Japan], and the like), polyvinyl pyrrolidone, polyvinyl alcohol, carboxymethyl-cellulose, lecithin, gelatin, and the like. Such emulsifying agents may be used either alone or in combination. The concentration of the emulsifying agent may be selected from the range of about 0.01% to about 20% and is preferably in the range of about 0.05% to about 10%.

The aforesaid evaporation of the solvent from the oil layer can be accomplished by conventional techniques. Thus, such evaporation is affected by gradual decrease of pressure under agitation with a propeller mixer or magnetic stirrer or by adjusting the degree of vacuum in a rotary evaporator. Higher stirring speed ensures smaller diameter of the product microcapsule. The time required for such procedures can be shortened by warming the W/O/W emulsion so as to make the solvent evaporation thorough, after the solidification of the polymer has progressed to some extent and the loss of the active ingredient from the first dispersed phase has decreased. When the thickening or solidification is effected by techniques other than temperature control, the evaporation may be effected by allowing the W/O/W emulsion to stand under stirring, warming the emulsion or blasting it with nitrogen gas. The process of evaporation of the solvent is an important process having great bearing on the surface structure of microspheres which governs the release of the active ingredient. For example, when the evaporation speed is increased, pits in the surface layer increase in number and size so that the release rate of the active ingredient is increased. The microspheres obtained in the above manner are recovered by centrifugation or filtration, and the free water-soluble active ingredient, emulsifying agents, etc. on the surface are removed by repeated washing with water, then, if necessary, the microspheres are warmed under reduced pressure to achieve a complete removal of moisture and of the solvent from the microcapsule wall. The above microspheres may be gently crushed and sieved, if necessary, to remove coarse microspheres.

Resuspension of the dried microspheres can be done in a solution of a cryoprotectant such as mannitol and bulk lyophilization of microsphere suspension in mannitol can be done in sterile trays. Shallow autoclavable trays such as Lyoguard trays from W. L. Gore & Company, USA may be used for this purpose. In one embodiment, these lyophilized microspheres may be aseptically powder filled into vials.

The particle size of microspheres depends on the desired degree of prolonged release. When they are to be used as a suspension, its size may be within the range satisfying the required dispersibility and needle pass requirements. For example, the average diameter may range from about 0.5 to 400 μm and preferably from about 2 to 200 μm. The microspheres according to this invention can be administered in clinical practice directly as fine granules or as formulated preparation. Thus, they can be used as raw materials for the production of final pharmaceutical preparations. Such preparations include, among others, injections, oral preparations (e.g., powders, granules, capsules, tablets, etc.), nasal preparations, suppositories (e.g., rectal, vaginal), and so on.

When the microspheres according to this invention are to be processed into an injectable preparation, they are dispersed in an aqueous vehicle together with a dispersing agent (e.g., Tween 80, HCO-60 (Nikko Chemicals), carboxymethylcellulose, sodium alginate, etc.), preservative (e.g., methyl-paraben, propyl-paraben, benzyl alcohol, chlorobutanol, etc.), isotonicity agent (e.g., sodium chloride, glycerin, sorbitol, glucose, etc.), etc. The vehicle may also be a vegetable oil (e.g., olive oil, sesame oil, peanut oil, cottonseed oil, corn oil, etc.), propylene glycol or the like. In this manner, a prolonged release injection can be produced. The prolonged release injection made from said microspheres may be further supplemented with an excipient (e.g., mannitol, sorbitol, lactose, glucose, etc.), redispersed, and then be solidified by freeze-drying or spray-drying, and on extemporaneous addition of a distilled water for injection or suitable vehicle for the reconstitution, such preparation gives a prolonged release injection with greater stability. When an injectable dosage form is employed, the volume of the suspension may be selected from the range of about 0.1 to 5 ml, preferably about 0.5 to 3 ml.

It was observed that in the process of preparing the microspheres of the present invention, the phase volume ratio of the primary emulsion to be formed is required to be adjusted to avoid the separation of PLGA/peptide complex gel phase in the primary emulsion and thus in the composition of the aqueous phase used for secondary emulsification. Without wishing to be bound by any theory, it was thought that the PLGA polymer with free end groups interacts with the peptide to form PLGA/peptide complex which behaves like a surfactant and stabilizes the internal aqueous droplets. The uniform water-in-oil (W/O) emulsion ranges from almost transparent to almost white optical nature depending on the phase ratio and type of polymer peptide. However, we have discovered that when hydrophobic peptides such as goserelin are used, these hydrophobic peptides could precipitate out of the aqueous solution as PLGA/peptide complex gel phase (as a third phase) during primary emulsification. Such precipitation was not observed in the case of hydrophilic peptides such as leuprolide or octreotide, under similar formulation and process conditions. Thus, when a hydrophobic peptide is used, the system consists of an aqueous internal phase, the polymer containing oily phase (both together in a form of an emulsion) and a phase separated peptide/polymer complex phase (other than emulsion), which affects the uniformity of the active ingredient distribution and encapsulation of active ingredient in microspheres. It was surprisingly observed that this problem could be solved by using an increased W/O phase ratio. By this way, the gel phase could be uniformly dispersed without any precipitate phase being formed in the primary emulsion thereby leading to uniform water-in-oil (W/O) emulsion and finally to microspheres having desired porosity and release profile.

We have observed that the dynamic transition temperature (dTg) of the PLGA polymer used in the microspheres or microcapsules prepared by the process of the present invention plays a significant role in deciding the product characteristics. The dTg is the temperature above which, the secondary, non-covalent bonds between the polymer chains become weak in comparison to thermal motion, and the polymer becomes rubbery and capable of elastic or plastic deformation, without fracture. The dTg of the PLGA polymer is low when the amount of residual solvent within the microspheres/microcapsules is high, and this dTg goes on increasing gradually as the solvent in the microspheres/microcapsules is gradually evaporated. It was surprisingly found that the temperature at which solvent evaporation is carried out can affect the physical as well as release characteristics of the microspheres/microcapsules. If the temperature is always maintained below the dTg of the PLGA polymer at any given point during the process of solvent evaporation, and gradually increased to the dTg, microspheres/microcapsules with desirable physical properties and release profile could be obtained. However, if the solvent evaporation is carried out at a temperature above the dTg of the polymer, or increased to a temperature above the dTg of the polymer, the microspheres/microcapsules were found not to have good physical characteristics, and had a slow release profile, at times releasing a maximum of only 70% of the active ingredient.

The microspheres of the invention have a volume mean diameter in the range of about 2 microns to about 200 microns. The preferred embodiments relate to microspheres having a volume mean diameter in the range of about 10 microns to about 50 microns.

Though the preferred route of administration of the lyophilized composition of octreotide acetate microspheres is by the intramuscular route, it may be administered by other routes such as the subcutaneous route.

The examples that follow do not limit the scope of the present invention and are merely used as illustrations.

### EXAMPLE 1

A sustained release injection composition of octreotide acetate was obtained as described in Table 1 below.

TABLE 1

| | |
|---|---|
| Ingredients | Quantity (mg/vial) |

Microsphere formulation

| | |
|---|---|
| Octreotide acetate | 11.2 |
| Purified gelatin | 4.0 |
| DL-lactic acid and glycolic acid copolymer | 188.8 |
| (3:1, molecular weight 10,000) | |
| Mannitol | 41.0 |

Formulation medium

| | |
|---|---|
| Sodium carboxymethyl cellulose | 10.0 |
| Mannitol | 12.0 |
| Water for injection | 2.0 |

Octreotide acetate was mixed with purified gelatin and the mixture was dissolved in water. The solution thus obtained was subjected to filtration, followed by lyophilisation of the solution to obtain a cake. This cake was dissolved in a sufficient amount of water for injection to obtain an aqueous phase. This aqueous phase was emulsified using a solution of the lactic acid-glycolic acid copolymer in methylene chloride, in a first tank, to obtain a primary emulsion. The primary emulsion was cooled to about 15° C. for about 30 minutes, and then pumped to a second tank containing an aqueous solution of mannitol and 0.1% polyvinyl alcohol. The mixture was homogenized to obtain a water/oil/water emulsion. The excess solvent was evaporated from this ternary emulsion, followed by sieving and drying of the microspheres. The dry microspheres are suspended in aqueous mannitol solution and lyophilized. The lyophilized microspheres were then filled into vials.

The lyophilized microspheres were then suspended in a formulation medium prior to administration, the medium comprising sodium carboxymethyl cellulose, mannitol and polysorbate 80 in sterile water for injection, the pH of the medium being adjusted with glacial acetic acid to about pH 5.0-6.0. The microspheres thus obtained were found to have a volume mean diameter of about 15.5 microns. The composition was found to provide an in vitro release profile as recorded in Table 2 below.

TABLE 2

| Time (days) | % drug released from microspheres |
|---|---|
| 1 | 27.22 |
| 14 | 56.91 |
| 28 | 77.70 |
| 35 | 96.03 |

The composition was also subjected to in vivo studies in rats to estimate the inhibition of growth hormone by octreotide acetate. The study was done on male Wistar rats of weight 203-218 gm.

The animals were randomized a day prior to the experiment; feed and water were given ad libium. The animals were divided into groups of ten per formulation (n=10): the Reference Formulation (Sandostatin®), the Test formulation (examples of this invention), the Placebo control, and the saline treated groups. The animals were anesthetized with thiopentone (36 mg/kg, i.p; dose volume 1 ml/kg). Blood for zero hour was collected from the retro orbital plexus of the animals, under anesthesia. Immediately after blood collection placebo/test formulations suspended in the supplied diluent were injected subcutaneously at a dose of 10 mg/kg (dose volume 2 ml/kg). Blood was collected under thiopentone anesthesia on days 2, 7, 14, 21, 28 and 35 from retro orbital plexus. The collected blood was centrifuged at 3000 rpm/40° C./10 mins for separating the plasma. Separated plasma was analyzed for Growth Hormone levels using EIA (Growth Hormone analysis kit from Cayman) and for Octreotide levels using LC/MS/MS.

The levels of growth hormone in the rats and the levels of octreotide in the plasma, estimated are recorded in Table 3 below.

TABLE 3

| | Growth hormone | % inhibition of | Plasma octreotide |
|---|---|---|---|
| Time (days) | levels (ng/ml) | growth hormone | levels (ng/ml) |
| 2 | 12.8 | 84.1 | 6.42 |
| 7 | 8.1 | 94.9 | 14.96 |
| 14 | 22.4 | 78.1 | 9.38 |
| 21 | 20.0 | 65.1 | 3.77 |
| 28 | 9.8 | 86.2 | 7.36 |
| 35 | 21.7 | 72.9 | 7.01 |

1. A sustained release microsphere composition comprising—
(i) microspheres comprising (A) a biodegradable polymer which is a homopolymer of lactic acid or a copolymer of lactic acid and glycolic acid having a monomer ratio of about 3:1, and (B) a therapeutically effective amount of octreotide acetate, and
(ii) pharmaceutically acceptable excipients,
which when injected, delivers octreotide acetate, for a period of at least one month.

2. A sustained release microsphere composition as in claim 1, wherein the biodegradable polymer used has an average molecular weight within the range of about 10,000 to about 20,000 Daltons.

3. A sustained release microsphere composition as in claim 1, wherein the biodegradable polymer is used in amounts ranging from about 70% to about 99% W/W of the microspheres.

4. A sustained release microsphere composition as in claim 1, wherein the microspheres have a volume mean diameter in the range of about 10 microns to about 50 microns.

5. A sustained release microsphere composition as in claim 1, wherein the composition is capable of delivering octreotide acetate over a period of about one month.

6. A sustained release microsphere composition as in claim 1, wherein the composition is capable of delivering octreotide acetate over a period of about three months.

7. A sustained release microsphere composition as in claim 1, wherein the composition is capable of delivering octreotide acetate over a period of about six months.

8. A sustained release microsphere composition as in claim 1, wherein the microsphere is suitable for intramuscular injection.

9. A sustained release microsphere composition as in claim 1, wherein the pharmaceutically acceptable excipient is mannitol.
